(19) United States
(12) Patent Application Publication
(10) Pub. No.: US 2003/0186259 A1
(43) Pub. Date: Oct. 2, 2003

(54) Method , kit and dna probes for detecting a fungal species in clinical material

(76) Inventors: J?uuml;rgen Löffler, (US); Hermann Einsele, T?uuml;bingen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/182,542

(22) PCT Filed: Jan. 18, 2001
(86) PCT No.: PCT/EP01/00537

(30) Foreign Application Priority Data
Jan. 28, 2000 (DE) 10003580.9

(51) Int. Cl. C12Q 1/68; C07H 21/04

(52) U.S. Cl. 435/6; 536/23.7

(57) ABSTRACT

Oligonucleotides of the sequence listing are used as DNA probes in a method and a kit for detecting a fungal species in clinical material, in order to detect via hybridization DNA segments of fungal DNA extracted from said clinical material.

The present invention relates to a method for detecting a fungal species in clinical material, containing the steps:

extracting fungal DNA from clinical material,

providing a DNA segment of the extracted fungal DNA, and

detecting the fungal species by hybridizing the DNA segment with specific DNA probes.

The invention further relates to DNA probes which can be used in said methods and also to a kit containing said DNA probes.

WO 97/07238 discloses a method of the abovementioned type. This publication likewise discloses hybridization probes which can be used to detect, inter alia, fungi of the species Candida albicans and Aspergillus fumigatus.

In recent years, invasive fungal infections have gained considerable importance as the major cause of morbidity and mortality in immunosuppressed patients. This is true, for example, for patients with bone marrow or organ transplants, chemotherapy patients, AIDS patients or patients with severe burns. For hematological patients in particular, aspergillosis has become the major cause of death.

Against this background, an early reliable diagnosis of a systemic fungal infection is indispensable for a suitable and successful therapy.

For this purpose, WO 97/07238, mentioned at the outset, describes a method which is based on the polymerase chain reaction (PCR hereinbelow) and in which first phagocytosed fungal cells are extracted from clinical material, preferably from whole blood, from which fungal cells the fungal DNA is then purified. A DNA segment from the fungal gene for 18ssu-rRNA is then amplified within the framework of a PCR with the aid of fungus-specific primers. This is based on the finding that said fungal gene has in various fungal strains and genera a sequence segment which on the one hand is flanked by two primer binding regions which are identical for all fungal strains and genera; on the other hand, however, the sequence of said segment is so different in the various fungal strains and genera that it can be used simultaneously for detecting the individual fungal species and genera.

The appropriately amplified DNA segment is then hybridized sequentially with various DNA probes which in each case are specific for the fungal species to be detected. In order to detect successful hybridization, the probes are labeled with digoxigenin using the transferase kit from Boehringer, Mannheim, and detection is carried out according to the Southern blot method using the usual color reaction.

Further hybridization probes which can be used in the known method are described in DE 196 35 347 C1.

Although the known method works very reliably and with high sensitivity using the known DNA probes, there is nevertheless a continued demand for methods which can be carried out more rapidly and/or more easily. One disadvantage of the known method and the known probes is the fact that the amplified DNA segment must be incubated with the individual DNA probes in individual experiments sequentially and/or in parallel, and, although the hybridization result is to be checked in each individual case by standard methods, this is nevertheless time-consuming.

In this connection, a method for quantitative PCR has been disclosed in recent years, which can be carried out in the so called LightCycler™ from Roche Molecular Biochemicals. This method makes it possible to observe amplification of the PCR products in real time and on-line cycle by cycle. For this purpose, hybridization probes which bind specifically to the PCR products of interest are also added to the PCR solution, in addition to the polymerase, nucleotides, buffer solutions and primers. In this connection, two sequence-specific DNA probes are used, which are labeled with different dyes. The sequences of the two probes are selected such that they hybridize to the target sequences of the amplified DNA segment in such a way that the 3′ end of one of the probes is located close to the 5′ end of the other probe, thereby bringing the two dyes into close proximity. The donor dye, for example fluorescein, is excited by a short-wavelength light source and emits fluorescent light at a slightly longer wavelength. If the two dyes are close to one another, the emitted energy excites the acceptor dye which is located on the second hybridization probe and which emits a fluorescent light at a different wavelength.

This energy transfer which is also referred to as fluorescence resonance energy transfer (FRET) depends very strongly on the distance between the two dye molecules. Energy is transferred efficiently only if the molecules are in immediate proximity (between 1 and 5 nucleotides), the extent of fluorescence being directly proportional to the amount of target DNA, which is generated during the PCR process.

As a result of the above, the acceptor dye on the second hybridization probe emits fluorescent light only if both hybridization probes have hybridized to the target sequence. As long as the two hybridization probes are in solution, only a very low background fluorescence can be measured.

In addition, the two hybridization probes do not impair the PCR process itself because said hybridization probes in each case detach again from the target DNA when, after the annealing step, the temperature is raised, in order to complete the next complementary strand. After such a replication cycle, the resulting double strand is melted, and in the next annealing step both the primers and the hybridization probes can anneal again to the target DNA.

As a result of this, the increase in DNA produced can be monitored on-line in the LightCycler™ via an increase in the fluorescence signal, using the method just described.

A combination of the two methods described thus far, i.e. of PCR in the LightCycler™ with the detection method according to WO 97/07238, would therefore have an advantage compared to the previously known detection method for fungal species in that it would be possible to provide the DNA segments from the 18ssu-rRNA gene and to hybridize them with the DNA probe in a single solution and in a single reaction, possibly resulting in distinct time advantages.

Against this background, it is the object of the present invention to provide for the method mentioned at the outset DNA probes which make it possible to carry out the known method using an embodiment such as, for example, in the LightCycler™.

According to the invention, this object is achieved by the oligonucleotides SEQ ID NO: 1-4 of the attached sequence listing. In this connection, the invention comprises not only the oligonucleotides SEQ ID NO: 1-4 but also oligonucleotides which hybridize with one of these oligonucleotides and also oligonucleotides which hybridize with an oligonucleotide hybridizing with such an oligonucleotide. DNA probes which can be used are not only the oligonucleotides having the sequences SEQ ID NO: 1-4 but also the complementary sequences thereof and slight modifications which do not adversely affect hybridization of said oligonucleotides with the DNA segment.

In this connection, oligonucleotides having the sequence SEQ ID NO: 1 and SEQ ID NO: 2 and also corresponding complementary and modified sequences are to be used for detecting the fungal species Aspergillus fumigatus, and the oligonucleotide sequences SEQ ID NO: 3 and SEQ ID NO: 4 and also the complementary and modified sequences are used for detecting the fungal species Candida albicans.

In a specific embodiment, the oligonucleotide having the sequence SEQ ID NO: 1 is labeled at the 5′ end with an acceptor and the oligonucleotide having a sequence SEQ ID NO: 3 is labeled at the 3′ end with a donor dye. In corresponding fashion, the oligonucleotide having the sequence SEQ ID NO: 3 is labeled at its 5′ end with an acceptor dye and the oligonucleotide having the sequence SEQ ID NO: 4 is labeled at its 3′ end with a donor dye. In the respective complementary sequences, the acceptor dye is located on the 3′ end and the donor dye is located on the 5′ end.

The inventors of the present application have noticed that the DNA probes described thus far make it possible to detect Aspergillus fumigatus and Candida albicans in a single PCR experiment, for example in the LightCycler™. If the acceptor dyes of the DNA probes for Aspergillus fumigatus and Candida albicans are different, both fungal species can be detected or discriminated against one another and quantified in a single experiment.

However, the novel DNA probes can be used not only for the LightCycler™ but, in the case of a “conventional” PCR, also enable a more rapid and easier detection of the particular fungal species than has been possible in the prior art. In fact, the only requirement in this connection is to add, after amplifying the DNA segments, the corresponding DNA probes and then to measure in a fluorimeter, whether a fluorescence signal of the acceptor dye or dyes is detectable at the excitation wavelength of the donor dye. Here, too, discrimination between Aspergillus fumigatus and Candida albicans is possible.

Against this background, the object on which the invention is based is achieved in the method mentioned at the outset by using as DNA probes one or more of the oligonucleotides of the invention, preferably providing by means of a PCR the DNA segment in an amount sufficient for detection.

The novel DNA probes may also be used, of course, for detection methods in which the DNA segments are provided not by means of PCR but, for example, by cloning or other methods.

In this connection, the invention also relates to a kit for detecting a fungal species, with said kit containing at least one of the oligonucleotides of the invention as DNA probe.

Further advantages arise from the following description. It goes without saying that the abovementioned features and the features still to be illustrated below can be used not only in the combinations indicated in each case but also in other combinations or on their own, without leaving the scope of the present invention.

The following examples illustrate the entire method for detecting fungal species in clinical material.

### EXAMPLE 1

### DNA Extraction

DNA extraction is carried out as described in Löffler et al., Med. Mycol. 36 (1998), pages 275-279:

The erythrocytes are lysed by incubating 5 ml of EDTA-anticoagulated blood in 15 ml of a hypertonic solution (10 mM Tris, 5 mM MgCl₂, 10 mM NaCl). This is followed by lysing the leukocytes in 1 ml of lysis buffer (10 mM Tris, 10 mM EDTA, 50 mM NaCl, 0.2% SDS, 200 μg/ml proteinase K). After appropriate centrifugation, the sediment now contains the released phagocytosed fungal cells.

The sediment is taken up in 500 μl of buffer which contains recombinant lyticase (1 U/100 μl) and incubated at 37° C. for 45 min in order to generate spheroblasts. Besides lyticase, the buffer contains 50 mM Tris, 1 mM EDTA and 0.2% β-mercapto-ethanol.

The spheroblast is lysed and protein is precipitated by using the QIAmp tissue kit from Qiagen, Hilden, Germany, according to the manufacturer's protocol.

The eluted DNA is taken up in 100 μl of elution buffer and used immediately for the PCR or stored at −80° C.

### EXAMPLE 2

### PCR in the LightCycler™

With the aid of two fungus-specific primers which have already been described in the initially mentioned WO 97/07238, a DNA segment of the extracted DNA is then amplified and simultaneously detected and quantified in the LightCycler™.

The PCR is carried out in glass capillaries which are heated to the appropriate reaction temperatures by the air flowing around said capillaries. The already mentioned primers (5′-ATT GGA GGG CAA GTC TGG TG and 5′-CCG ATC CCT AGT CGG CAT AG) bind to conserved regions of the 18ssu-rRNA fungal gene and cause amplification of an approx. 500 base pair DNA segment which can be detected with the aid of two DNA probes labeled with different dyes.

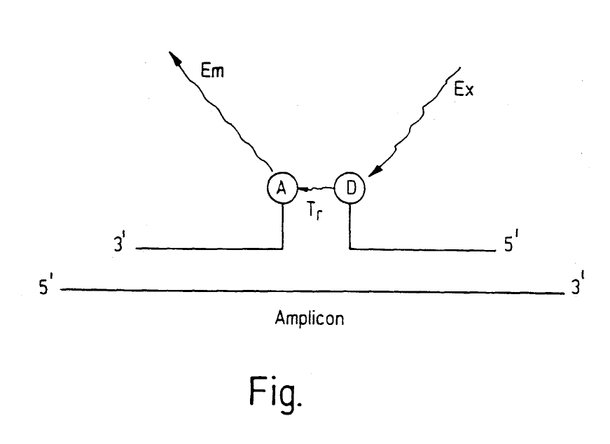

FIG. 1 shows such a DNA segment which is referred to there as amplicon.

Above said amplicon, two DNA probes are shown, the left probe of which is labeled at its 5′ end with an acceptor dye A and the right probe of which is labeled at its 3′ end with a donor dye D. The sequences of the two DNA probes are selected such that the dyes A and D are only one to five nucleotides apart.

The dye D is then excited at an appropriate wavelength Ex, it emits light of a wavelength Tr, which leads to excitation of the dye A which, as a result, emits at a wavelength Em. If, due to excitation at the wavelength Ex, an emission of wavelength Em can be detected, this consequently means that both DNA probes are hybridized to the amplicon and, as a result, said amplicon cannot only be detected but also be quantified in the reaction solution, because the intensity of the fluorescence signal at wavelength Em increases with the amount of amplicon present in said solution. By calculating back to the starting point of the PCR reaction, it is possible to infer in a manner known per se the amount of the original amplicon in the starting solution or of the extracted DNA from the exponential course of the fluorescence signal via the number of cycles. For this purpose, an external standard of genomic fungal DNA is used, which is comeasured in dilution series.

The PCR solution contained Taq polymerase, LightCycler™ hybridization 1×reaction buffer, dNTP mix, 3 mM MgCl₂ and 12.5 pmol of primer. The amplicon was detected by using the LightCycler™ DNA master hybridization probes kit according to the manufacturer's instructions.

The fungal species Aspergillus fumigatus was detected by using the following DNA probes:

5′-TGA GGT TCC CCA GAA GGA AAG GTC CAG C (SEQ ID NO: 1), labeled at the 5′ end with the acceptor dye LightCycler™ Red 640, and

5′-GTT CCC CCC ACA GCC AGT GAA GGC (SEQ ID NO: 2), labeled at the 3′ end with the donor dye fluorescein.

Candida albicans was detected by using the following DNA probes:

5′-TGG CGA ACC AGG ACT TTT ACT TTG A (SEQ ID NO: 3), labeled at the 5′ end with LightCycler™ Red 640, and

5′-AGC CTT TCC TTC TGG GTA GCC ATT (SEQ ID NO: 4), labeled at the 3′ end with fluorescein.

32 samples were processed in parallel using in each case 45 cycles of denaturation (1 sec at 95° C.), annealing (15 sec at 62° C.) and enzymatic chain extension (25 sec at 72° C.). The PCR run required 45 min.

### EXAMPLE 3

### Results

The sensitivity of the method according to Examples 1 and 2 proved to be the same as in the known method according to WO 97/07238, and it was possible to detect 5 CFU/ml of Candida albicans and Aspergillus fumigatus, respectively, fungal cells seeded. The assay showed high reproducibility of 96-99% and was linear in a region between 10¹ and 10⁴ conidia.

Experiments with clinical material of patients with hematological malignancies and histologically detected invasive fungal infection were likewise successful. Five of nine positive samples showed a fungal load of between 5 CFU/ml and 10 CFU/ml, two of the nine samples of between 10 CFU/ml and 100 CFU/ml and two last samples showed a fungal load of more than 100 CFU/ml.

If the probe SEQ ID NO: 1 or SEQ ID NO: 3 is labeled with a dye emitting at a different wavelength, for example LightCycle™ Red 705, Candida albicans and Aspergillus fumigatus can be detecteded in a single reaction by measuring Em both at 640 nm and at 705 nm.

### SEQUENCE LISTING

<110> Ebarhards-Karls-Universität Tübigen Universitätsk

<120> DNA probes for detecting fungal species

<130> 5402P191

<140>

<141>

<160> 4

<170> PatentIn Ver. 3.1

<210> 1

<211> 28

<212> DNA

<213> Artificial sequence

<220>

<223> Description of the artificial sequence: DNA probe for A. fumigatus

<400> 1

tgaggttccc cagaaggaaa ggtccagc

28

1. An oligonucleotide having the nucleotide sequence SEQ ID NO: 1 from the attached sequence listing:
TGAGGTTCCC CAGAAGGAAA GGTCCAGC.

2. An oligonucleotide having the nucleotide sequence SEQ ID NO: 2 from the attached sequence listing:
GTTCCCCCCA CAGCCAGTGA AGGC.

3. An oligonucleotide having the nucleotide sequence SEQ ID NO: 3 from the attached sequence listing:
TGGCGAACCA GGACTTTTAC TTTGA.

4. An oligonucleotide having the nucleotide sequence SEQ ID NO: 4 from the attached sequence listing:
AGCCTTTCCT TCTGGGTAGC CATT.

5. An oligonucleotide which hybridizes with an oligonucleotide as claimed in any of claims 1 to 4.

6. An oligonucleotide which hybridizes with an oligonucleotide as claimed in claim 5.

7. The use of the oligonucleotides of claims 1 and 2 for detecting Aspergillus fumigatus.

8. The use of the oligonucleotides of claims 3 and 4 for detecting Candida albicans.

9. A method for detecting a fungal species in clinical material, comprising the steps:
extracting fungal DNA from the clinical material,
providing a DNA segment of the extracted fungal DNA, and
detecting the fungal species by hybridizing the fungal segment with specific DNA probes,
characterized in that the DNA probes used are one or more of the oligonucleotides as claimed in any of claims 1 to 6.

10. The method of claim 9, characterized in that the DNA segment is provided by means of a PCR in an amount sufficient for detection.

11. A kit for detecting a fungal species, characterized in that it comprises at least one of the oligonucleotides as claimed in any of claims 1 to 6 as DNA probe.


SEQUENCE LISTING (4 sequences)

SEQ ID NO: 1 - DNA, 28 bp, Artificial Sequence
  DNA probe for A. fumigatus
tgaggttccccagaaggaaaggtccagc

SEQ ID NO: 2 - DNA, 24 bp, Artificial Sequence
  DNA probe for A. fumigatus
gttccccccacagccagtgaaggc

SEQ ID NO: 3 - DNA, 25 bp, Artificial Sequence
  DNA probe for C. albicans
tggcgaaccaggacttttactttga

SEQ ID NO: 4 - DNA, 24 bp, Artificial Sequence
  DNA probe for C. albicans
agcctttccttctgggtagccatt